(19) United States
(12) Patent Application Publication
(10) Pub. No.: US 2006/0014260 A1
(43) Pub. Date: Jan. 19, 2006

(54) Lower cellulase requirements for biomass cellulose hydrolysis and fermentation

(76) Inventors: Zhiliang Fan, Hanover, NH (US); Xiongjun Shao, West Lebanon, NH (US); Lee R. Lynd, Meriden, NH (US)

Correspondence Address: LATHROP & GAGE LC, 4845 PEARL EAST CIRCLE, SUITE 300, BOULDER, CO, 80301, US

(21) Appl. No.: 11/124,941

(22) Filed: May 9, 2005

Related U.S. Application Data
(60) Provisional application No. 60/569,346, filed on May 7, 2004.

(51) Int. Cl. C12P 7/06

(52) U.S. Cl. 435/161

(57) ABSTRACT

A semi-continuous simultaneous saccharification and fermentation (SSF) process for the bioconversion of cellulose into ethanol and other organic chemicals is disclosed. The process provides for substantially higher substrate conversion at a given enzyme loading (or alternatively a lower enzyme loading used to achieve the same conversion) in a reactor operated according to a semi-continuous feeding protocol.

### RELATED APPLICATIONS

This application claims the benefit of priority to U.S. provisional patent application Ser. No. 60/569,346, filed May 7, 2004, which is incorporated by reference herein.

### GOVERNMENT RIGHTS

The U.S. government has certain rights in this invention as provided for by the terms of Grant No. 60NANB1D0064, awarded by the National Institute of Standards and Technology.

### BACKGROUND

Production of ethanol from cellulosic biomass has been the subject of an intense R&D effort for several decades, yet the saccharification and fermentation technologies are not ready for widespread commercialization. One of the key reasons for this problem is the difficulty that is associated with hydrolyzing cellulose to simple sugars for fermentation. Process steps associated with overcoming the recalcitrance of cellulosic biomass are generally the most costly and have the greatest potential for R&D-driven improvement. Cellulose recalcitrance is typically overcome by acid pretreatment followed by enzymatic breakdown of the pretreated cellulose by cellulase enzymes. The high current cost of enzymes presents an obstacle to making bioconversion products commercially viable competitors to traditional fossil fuels. Cost estimates for suitable cellulase enzymes currently range from $0.30-0.50/gallon of ethanol that costs about $1.16 to $1.46/gallon. Significant research efforts are underway, by Genencor and others, to produce lower cost cellulase enzymes for hydrolysis. While this research proceeds and the cost of enzymes remains a limiting factor in the production of ethanol from cellulosic biomass, any means of reducing the amount, and therefore the cost, of these enzymes will remain an important commercial goal.

U.S. Pat. Nos. 5,258,293 and 5,837,506 issued to Lynd et al. are incorporated herein by reference. These patents show continuous reactor processes for saccharification and fermentation processes, and discuss a variety of reactor configurations. Among the process options for producing ethanol from lignocellulosic substrates (e.g., trees, grasses, and solid wastes) is Simultaneous Saccharification and Fermentation (“SSF”), which utilizes two microbial systems, one of which produces cellulase enzymes and the other of which carries out the fermentation process to produce ethanol. SSF may also be practiced with purified enzymes used in place of, or in addition to, microorganisms that produce cellulase enzymes.

In known batch processes for producing ethanol, reactants are added to a reaction vessel at the beginning of the production cycle and ethanol product is withdrawn from the vessel at the end of the production cycle, with no intermediate addition of raw materials or withdrawal of product from the vessel. In such batch processes the rate of ethanol production can be limited by the existence of large amounts of hydrolysis products (glucose) and final product (ethanol) and low initial concentrations of microorganisms. In addition, the productivity of batch processes inherently suffers from “down time” during which equipment is cleaned and the bioreactor is recharged. Such rate limitations give rise to a need to use larger bioreactors for a given rate of ethanol production.

A continuous stirred tank reactor (CSTR) process overcomes at least some of the limitations of batch processes. The CSTR process features continuous stirring or agitation of the substrate slurry by, for example, mechanical mixing or liquid recycling. The CSTR process allows optimization and balancing of the hydrolysis and fermentation rates to eliminate the large accumulation of glucose and the resulting inhibition of ethanol production. The CSTR process employs continuous addition of fermentable substrate, catalysts and fermentation agents, and continuous removal of any residual substrate—and product—containing broth. The CSTR process has perpetually high concentrations of microorganisms, much reduced down time compared to batch reactors, generally lower maximum concentrations of potentially inhibitory mono- and disaccharides, but higher ethanol concentration. Thus, the relative merits of batch and CSTR will depend upon the needs and circumstances surrounding a given application.

The use of a continuous solids retaining bioreactor (CSRB) provides further improvements in the production of ethanol. The CSRB improves productivity and yield by providing differential solids retention and thus increasing the concentration of substrate particles in the reactor and increasing the hydrolysis rate. The use of a CSRB increases the overall hydrolysis rate and thus reactor productivity by maximizing the amount of cellulose/enzyme complex in the reactor. The key to efficiency in the CSRB process appears to be the management and control of the cellulose/enzyme complex in the reactor.

A further advancement in the production of ethanol is the use of cascaded CSRBs, in which the output from one CSRB reactor vessel becomes the input feed to the next CSRB reactor vessel. This arrangement overcomes the problem of decreased or limited productivity enhancement with high conversion, as the cascaded reactors achieve higher total conversion for an equal cumulative residence time. However, the solids retention in the later stages is always less than in the early stages as a result of reduced cellulose particle size, because smaller particles require more time to settle. An advantage of the cascaded CSRB system over the single CSRB is that at high conversion, the presence of large amounts of ethanol in a single CSRB inhibits the further production of ethanol, whereas this inhibition is alleviated to some extent in a cascade system because the average concentration of alcohol seen by the reaction is reduced as the reaction proceeds through sequential steady state reactors at increasing ethanol concentration until the final concentration is reached.

Most process design and evaluation work has anticipated either batch reactors, which are not fed after being initially charged with substrate, or fully continuous reactors, which are charged with substrate at a constant rate. The highest cellulose conversion at a given enzyme loading is expected in a batch reactor, although several general features of batch reactors may diminish their attractiveness compared to fully continuous reactors. For example, in the case of solid feedstocks, mixing energy requirements are typically greatest for unreacted material, and thus for batch reactors. Additionally, batch systems require lost production time and increased costs related to emptying, filling and sterilization.

### SUMMARY

The instrumentalities reported herein overcome the problems that are outlined above and advance the art by providing a semi-continuously operated system that suffers less production-related costs than do batch or fully-continuous reactors. This system reduces costs by using lower enzyme loading concentrations to achieve a given conversion efficiency relative to a fully continuous process, thus overcoming major disadvantages of known systems.

In one aspect, a substantially higher substrate conversion may be obtained at a given enzyme loading (or alternatively a lower enzyme loading used to achieve the same conversion) in a reactor that is operated semi-continuously rather than fully continuously. Moreover, the reduction of required cellulase loading that accompanies semi-continuous feeding becomes more pronounced as the feeding frequency is reduced. As used herein, the term “feeding frequency” is defined as the number of feedings per residence time. An optimum semi-continuous process may exist for different substrates, with the optimum parameters determined by lower enzyme costs overall but with greater costs for mixing and feed storage as the feeding frequency decreases relative to a fully continuous process. This balance of costs results in a lower overall process cost.

More specifically, and as described below, it has been determined that the amount, and therefore the cost, of added commercial cellulase enzymes that are needed to hydrolyze cellulose in paper sludge can be significantly lowered by reducing both the feeding frequency and enzyme dosing levels in a simultaneous saccharification and fermentation process.

Paper sludge is one viable feedstock for ethanol production. Paper sludge is solid residue arising from pulping and paper-making, and is typically removed from process wastewater in a primary clarifier. At a disposal cost of $30/wet ton, the cost of sludge disposal equates to $5/ton of paper that is produced for sale. The costly alternative of disposing wet sludge at this price is a significant incentive to convert the material for other uses, such as conversion to ethanol. The presently described paper sludge process is also applicable to such other cellulosic biomass feedstocks as pretreated corn stover, wood chips or grass. The saccharification and/or fermentation products may be used to produce ethanol or higher value added chemicals, such as organic acids, aromatics, esters, acetone and polymer intermediates.

Ethanol production is accomplished according to one embodiment by a semi-continuous feeding protocol. This process has been found to be an efficient and productive means for ethanol production where both feeding frequency and enzyme loading are reduced by operation of a bioreactor in a semi-continuous manner. The process offers the advantage of potentially being more cost efficient than both batch and continuous bioreactor processes.

In one aspect, a bioreactor may be charged with a slurry of chopped solid biomass (substrate), enzymes, growth media and one or more types of microorganisms. The bioreactor is semi-continuously fed solid, cellulosic substrate and a liquid input of enzymes and growth media at a pre-determined and optimized frequency until near theoretical hydrolysis is achieved.

Additional advantages include, for example, the ability to use a wide variety of fermentable substrates, including waste products that might otherwise have been unusable; the ability to increase the productivity of ethanol-producing reactors and thus increase yield at a given enzyme loading; and the ability to decrease the costs of ethanol production, possibly leading to an increased use of ethanol as an alternative fuel source.

### BRIEF DESCRIPTION OF THE DRAWINGS

Systems and methods will be described in further detail with reference to the following detailed description and the accompanying drawings, in which:

FIG. 1 is a schematic diagram of a semi-continuous, solids-fed reactor system according to one embodiment;

FIG. 2 is a graphical representation of the conversion of sludge waste over time according to one embodiment; and

FIGS. 3 and 4 are graphical representations of conversion versus feeding frequency according to the description below.

### DETAILED DESCRIPTION

There will now be shown and described a solids-fed bioreactor that is semi-continuously fed with cellulosic material, enzyme and growth media to produce ethanol. The result of operating a bioreactor according to a semi-continuous feeding protocol is a significant decrease in suitable enzyme loading per volume of the fermentable sugars that are produced by hydrolysis. This is alternatively described as an economically appreciable increase in the efficiency of converting cellulose into fermentable sugars with a constant enzyme loading relative to a fully continuous system. Overall, the process reduces the costs that are associated with bioconversion of cellulose into ethanol and other fermentation products.

In one embodiment, predetermined quantities of materials are added to a bioreactor to form a slurry. The materials may include a cellulosic substrate material, an enzyme for hydrolyzing the cellulose to simple sugars, one or more bacterial or fungal organisms for fermenting the sugar to ethanol, and a growth medium to sustain the viability of the organisms. The raw materials in the bioreactor are then reacted at an optimal temperature, such as approximately 37° C., to promote and maintain hydrolysis of the cellulosic substrate and fermentation of the resulting hydrolysis products.

The raw materials for ethanol production may be added to a reaction vessel to form a slurry. The slurry may be agitated so that the solid cellulosic substrate particles are uniformly dispersed within the reactor vessel to ensure that they are exposed to cellulase. Agitation may be accomplished using a variety of devices, including mechanical devices such as vortex mixers and gate stirrers. A suitable agitation speed using one particular type of stirrer is about 100 rpm, although the system may also be operated at higher or lower agitation speeds, or with no agitation at all. Agitation may be provided continuously or periodically for a period of time such as from about 30 minutes to several hours or days.

At various times, it may be desirable to cease agitation to remove ethanol-containing effluent from the reactor vessel. Ethanol-containing effluent may be removed from a top portion of the reactor vessel, since the top portion of the slurry should be relatively free of suspended solids after agitation has ceased for a short period. In one embodiment, ethanol may be removed from the reaction vessel just prior to additional substrate and liquid components being added. The timing and protocols for ethanol removal will depend on several factors including, for example: (1) the optimized feeding frequency, (2) tolerance of microorganisms to high ethanol concentrations, (3) volume limitations of the reactor vessel and (4) energy requirements of starting and stopping the mechanical components of the system. One skilled in the art can readily determine the optimum schedule for removal of products.

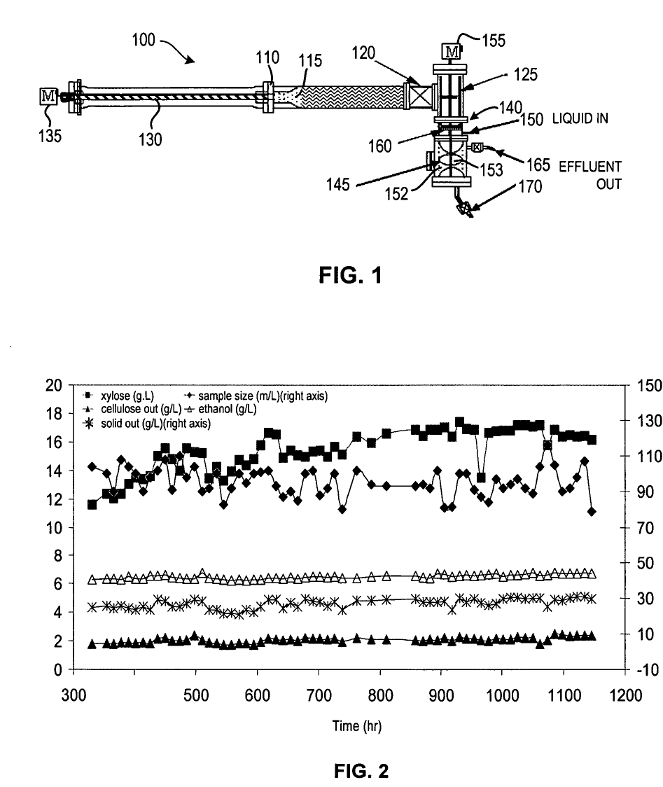

FIG. 1 illustrates a bioreactor 100. A feed tube 110, containing a plug of solid waste 115, is connected by an isolation valve 120 to a chopper 125. The plug of solid waste 115 is preferably paper sludge, but may be another type of cellulosic or lignocellulosic biomass. The isolation valve 120, which may be a ball valve, allows for replacement of feed tube 110 with sterility maintained by steaming-in-place. The plug of solid waste 115 is advanced through the isolation valve 120 into the chopper 125 at a rate determined by the number of rotations of a screw 130 driven by a timer-controlled motor 135. When the plug of solid waste 115 encounters the chopper 125, sludge is sheared off and falls through a connecting unit 140 into an agitator 145. Liquid reagents are added to the agitator 145 through tube 150 which passes through the wall of the connecting unit 140. The resultant slurry 152 formed in the agitator 145 may be stirred by a mechanical mixer 153 driven by motor 155 which is supported by bearing 160. Effluent may be removed from the bioreactor through conduits 165, 170. The mixing speed or cycle within agitator 145 may be adjusted so that, at appropriate times, gravity segregation occurs. Effluent conduit 165 may be used to withdraw ethanol-containing liquid effluent from the top of the agitator 145, whereas conduit 170 may be used to withdraw residual solids from the bottom of the agitator 145.

Substrate

The terms “substrate”, “cellulosic material”, “biomass”, “cellulose” and “solids” may be used interchangeably herein and shall be understood to refer to bulk organic materials upon which enzymes may act to release simple sugars. In one embodiment, the cellulosic material is a hemicellulose comprised of polysaccharides including glucans, mannans and xylans.

Substrates used in practice are generally categorized as lignocellulosic raw materials. Exemplary classes of lignocellulosic raw materials which may be used as substrates include woody biomass, herbaceous biomass (e.g., forage grasses, herbaceous energy crops), agricultural residue and waste material (e.g., waste paper sludge and municipal solid waste). Exemplary woody biomass materials include hardwoods such as poplar, oak, maple and birch.

The size range of the substrate material varies widely and depends upon the type of substrate material used as well as the requirements and needs of a given process. Depending on the pretreatment process employed and the size of the substrate particles prior to pretreatment, substrate material ranges from less than a millimeter to inches in diameter, and need only be of a size that is reactive. The particle size of the substrate material after pretreatment is in the range of about 0.5 to 12 millimeters, and typically measures about 2 millimeters.

The substrate material may be pretreated. Exemplary pretreatment processes include dilute-acid hydrolysis, steam explosion, and ammonia fiber explosion. The cellulose can be pretreated by heating it in, for example, a dilute aqueous sulfuric acid solution (0.45%) at a temperature of at least 160° C. for up to several minutes. The pretreated cellulose can then be sterilized, if desired, to prevent growth of other microorganisms during the fermentation reaction.

Microorganisms

A variety of microorganisms are known to be useful for the conversion of organic material to ethanol. Examples of microorganisms which may be used in practice are fermentation agents, such as Saccharomyces cerevisiae for producing ethanol. An alternative ethanol-producing organism which may be used is Zymomonas mobiles or a member selected from the Zymomonas, Erwinia, Klebsiella, Xanthomonas or Escherichia genii. Other microorganisms that convert sugars to ethanol include species of Schizosaccharomyces (such as S. pombe), Pichia (P. stipitis), Candida (C. shehatae) and Pachysolen (P. tannophilus). One skilled in the art can readily identify a variety of additional suitable microbial systems which may be used.

Microorganisms that may be used to produce cellulase enzymes either in vitro or in situ include Trichoderma reesei, Acidothermus cellulyticus and Trichoderma koningii. In one embodiment, enzymes are produced in vitro and purified before addition to the slurry.

A particular enzyme useful for saccharification is Genencor CL cellulase available from Genencor Inc. (San Francisco, Calif.) combined with Novozyme 188 β-glucosidase available from NOVO Laboratories Inc. (Wilton, Conn.). The addition of β-glucosidase to the cellulase increases the specific activity of the cellulase solution by reducing the accumulation of cellobiose and preventing or minimizing the resulting inhibition of glucose production.

Conditions for cellulase hydrolysis are typically at temperatures between about 30° C. and 60° C. and a pH between about 4.0 and 8.0. In one embodiment, conditions include a temperature between about 30° C. and 48° C. and a pH between about 4.0and 6.0.

Growth Media

A variety of suitable growth media are well known in the art and can be selected by one having ordinary skill for the particular microorganism(s) used. Generally, it is required that a suitable growth medium be able to provide the chemical components necessary to maintain metabolic activity and to allow cell growth.

Feeding Frequency

The term “feeding” shall refer to addition of substrate, enzymes and liquid components to an operating bioreactor. An initial feeding also includes addition of fermentation microorganisms. Solid substrate is preferably added by progressing a solid plug through a chopper. Liquid components and enzymes may be added by an inlet valve. Liquid components may include water, nutrients, growth media, buffering agents and the like.

The term “feeding frequency” shall apply to reactors fed over an extended or indefinite period of time. Feeding frequency refers to the number of feedings per liquid residence time. For example, if one reactor volume of new material is added every 4 days, the residence time is 4 days. If the reactor is fed twice a day (i.e., every 0.5 days), the feeding frequency is 4/0.5=8. The reaction time for one reactor volume of material is usually about 24-144 hours, and typically 24-96 hours. An optimum feeding frequency is achieved when an economic advantage is realized relative to both batch and continuous processes. In an semi-continuous solids-fed bioreactor system, an optimum feeding frequency involves cost savings related to decreased “down time” relative to batch processes and decreased enzyme loading relative to continuously-fed systems.

The working examples below illustrate the disclosed process by way of example and not by limitation.

### EXAMPLE 1

### Exemplary Process

A small-scale SSF reactor as shown in FIG. 1 was operated according to a semi-continuous feeding protocol. An agitator fitted with a bottom mounted stirrer was autoclaved to ensure sterility. After autoclaving, the agitator was filled to 10% full volume with growth medium which had been pH adjusted to 4.5 using sulfuric acid. The agitator was then brought to a temperature of 37° C. by a water jacket surrounding the agitator, and an inoculum of microorganisms was introduced by aseptic technique. The organism was Saccharomyces cerevisiae which had been grown overnight on a 10 g/L yeast extract, 20 g/L peptone, 20 g/L glucose solution. Following inoculation, semi-continuous feeding of the reactor commenced using 86.5 g/L waste paper sludge as the cellulosic substrate and addition of cellulase at a loading of 20 IU/g cellulose. Substrate entered the reactor through a chopper and was allowed to fall into the agitator. The slurry was stirred using a gate stirrer at 100 rpm. Temperature of the reactor was maintained at 37° C. by the use of a recirculation water bath. The pH of the reactor was held at 4.5 by the addition of dilute sulfuric acid. The reactor was operated at a 4 day nominal residence time and fed every 12 hr, corresponding to a feeding frequency, ƒ, of 8 (ƒ=feedings per residence time). Samples were taken at the end of each 12 hr feeding cycle, and the reactor was said to be at steady-state when the composition of these samples exhibited a constant trend with time.

Under the conditions tested, the steady-state output cellulose concentration was 6.63 g/L, representing 92% conversion. The effluent ethanol concentration was 42.2 g/L, representing a yield on cellulose of 46.6% (91.4% of theoretical). Ninety four percent of the incoming paper sludge xylan was hydrolyzed, but was not fermented due the limitations of the fermenting organism used, S. cerevisiae. Steady-state ethanol concentrations up to 50 g/L have been achieved in other runs (data not shown). Ethanol concentrations in the range of 40 to 45 g/L are typical of process designs for pretreated cellulosic feedstocks. At this concentration, ethanol recovery is relatively inexpensive and does not prevent such processes from having a favorable overall energy balance. A semi-continuous solids-fed fermentation reactor system of the type shown in FIG. 1 was built and used to perform metered saccharification by aseptic feeding of paper sludge over an extended period of time.

As shown, in FIG. 2, the system achieved a stable steady-state for the entire study period with good material balance. FIG. 2 is a graphical representation of the conversion of sludge waste over time including the input sample size in mL, and process outputs of xylose, cellulose, total solids and ethanol. All values in FIG. 2 are determined with respect to the left axis, except when the right axis is specified.

### EXAMPLE 2

### Mathematical Modeling

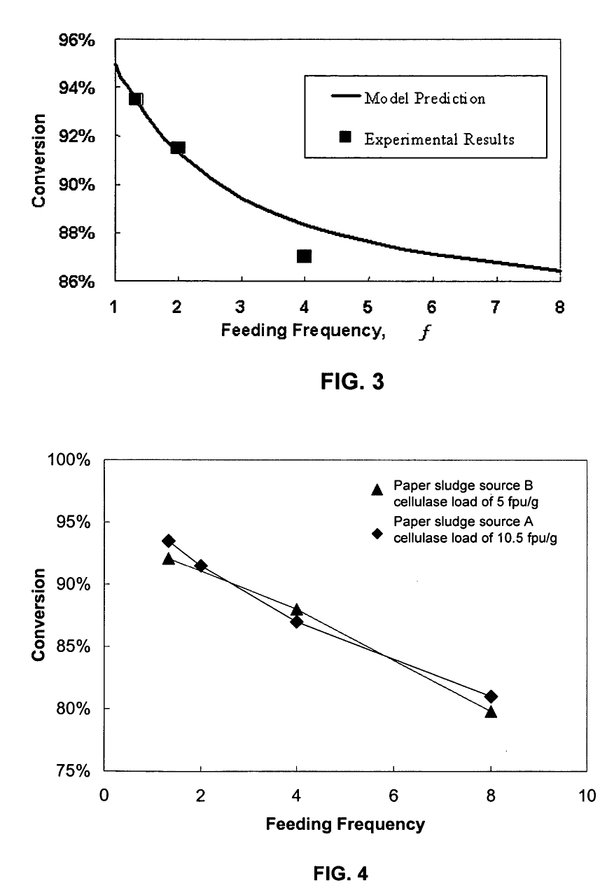

A mathematical model by South et al. (Enz. Microb. Technol., 1995) was modified to accommodate intermittent feeding according to the system design concepts illustrated in FIG. 1. The feed rate in the process of Example 1 was adjusted to various feeding frequencies. Comparative results were obtained on the basis of the predictive model and actual experimentation. The comparison resulted in a substantially identical overlay of the predictive and analytical results, as is shown in FIG. 3. The conversion efficiency diminished as feeding frequency increased; however, where the model predicted a flattening of slope curvature in the conversion efficiency beyond a feeding frequency of about 3, the actual results fell somewhat below this prediction. Nonetheless, the mathematical model remains a good predictive tool and shows that some predictive modeling may be used to predict the optimum conversion efficiency for a given type of biomass. This optimum is generally determined as a balance of costs. Decreasing the feeding frequency reduces costs by increasing the output of a plant facility. Increased conversion efficiency decreases overall costs by reducing incomplete conversion of the biomass feed. FIG. 3 shows that a given plant may be operated on the basis of either mathematical data or empirical data to reduce overall costs by decreasing the feeding frequency until a point of higher conversion efficiency improving overall profitability.

During the operation of the SSF reactor as described in Example 1 to convert, in this case, paper sludge to ethanol, aspects of the most efficient operation parameters were delineated. By optimizing enzyme loading in relation to feeding frequency, ƒ, a relationship of ƒ to conversion efficiency was determined. As shown in FIG. 3, conversion decreased asfincreased at constant enzyme loading. This trend was shown both for experimental data, and also by model predictions.

In particular, data for two paper sludges shows that conversion declined from about 92.5% to about 80% as the feeding frequency was increased from 1.33 to 8 (FIG. 4). For both sludges, approximately twice the enzyme loading was required to achieve conversion in the 90 to 95% range at ƒ=8 as compared to ƒ=1.33. Thus, the impact of the feeding frequency can be seen to roughly halve the required cellulase loading.

### EXAMPLE 3

### Comparative Cellulase Loading

FIG. 4 shows a comparison of two paper sludges where the cellulase load was decreased from a typical 15 fpu/g, as is consistent with minimum requirements for the prior art, to 10.5 or 5 fpu/g. Very high conversions were achieved at the decreased cellulase loading values. The finding that high conversion can be achieved at reduced enzyme loadings at low ƒ values is significant in light of the impact of the cost of cellulase enzymes on the process economics. For example, decreasing the feeding frequency, ƒ, directly decreases the amount (and cost) of enzymes added while reaping the significant benefit of improved conversion efficiency. Improving the efficiency of conversion of a biomass feedstock to final product provides a second significant benefit of increasing the amount of product produced per ton of feedstock processed, whether it is paper sludge, or other materials such as agricultural residues like corn stover or the like. This relationship will also be true for other chemical products besides ethanol since the conversion efficiency is related first to sugar yields and not final products from a sugar fermentation.

It was further determined that reducing the cellulase addition rate, measured as filter paper units for cellulases, from a typical 15 fpu/g cellulose to 10.5 fpu/g cellulose, and reducing the feeding frequency to 1.33 additions per residence time achieved a very high cellulose conversion efficiency of about 94% with paper sludge source A. Importantly, paper sludge source B permitted a further reduction of enzyme addition levels to 5 fpu/g cellulose and achieved approximately 93% conversion efficiencies. Both paper sludge materials showed nearly a straight line improvement of conversion efficiency with reduced frequency of addition whether at the 10.5 or 5 fpu/g cellulase dosing rates. Such high conversion efficiencies at these low enzyme dosing rates are expected to dramatically improve the economics of production of any fermentation chemical from a cellulosic biomass feedstock, whether it is ethanol from paper sludge as demonstrated here or another combination of biomass feedstock and chemical fermentation product.

### EXAMPLE 4

### Description of a Mathematical Model

The noticeable and desirable efficiency associated with operating a solids-fed bioreactor in a semi-continuous manner, rather than a fully continuous manner, can be better understood and demonstrated by modeling a semi-continuous system in a computational fluid dynamics (CFD) framework. In a model of enzymatic hydrolysis of cellulose, three factors must be taken into consideration:
1) an adsorption model should allow for either enzyme or substrate to be in relative excess;
2) declining specific activity of enzyme-cellulose complexes with increasing cellulose conversion;
3) for non-batch reactors, a particle population model should account for variation in rate of particles with different time in the reactor.

A dynamic adsorption model was used to calculate the concentration of cellulose-enzyme complex. For substrate population i, defined by a given discrete feeding event, the rate of enzyme adsorption to cellulose and lignin can be expressed as

$$\begin{matrix}{r}_{CE} ( i ) = {k}_{fc} [ {E}_{f} ] {σ}_{C} [ {C}_{f} ( i ) ] - \frac{{k}_{fc}}{{K}_{C}} [ CE ( i ) ] & ( 1 ) \\ {r}_{LE} = {k}_{fl} [ {E}_{f} ] {σ}_{L} [ {L}_{f} ] - \frac{{k}_{fl}}{{K}_{L}} [ LE ] & ( 2 )\end{matrix}$$

Where i is the index for an individual substrate population, and [CE(i)] (g/L) and [LE] (g/L) are the concentrations of cellulose-enzyme complex and lignin-enzyme complex respectively. [E_{ƒ}], [C_{ƒ}(i)] and [L_{ƒ}] are the concentrations of enzyme, cellulose and lignin that are not bound in units of g/L. ^{σ}_{C} and ^{σ}_{L} represent the respective capacity of cellulose and lignin to bind enzyme in units of g/g. k_{ƒc}(L g⁻¹h⁻¹) is the cellulose adsorption rate constant, K_{C} (L/g) is the cellulose equilibrium adsorption constant. k_{ƒl}(L g⁻¹h⁻¹) is the lignin adsorption rate constant, and K_{L} (L/g) is the lignin equilibrium adsorption constant.

Conservation equations for cellulose, lignin and enzyme are:

$$\begin{matrix}[ {C}_{f} ( i ) ] = [ C ( i ) ] - \frac{[ CE ( i ) ]}{1 + {σ}_{C}} & ( 3 ) \\ [ {L}_{f} ] = [ L ] - \frac{[ LE ]}{1 + {σ}_{L}} & ( 4 ) \\ [ {E}_{f} ] = [ E ] - \frac{{σ}_{C}}{1 + {σ}_{C}} {\sum }_{i = 0}^{n} [ CE ( i ) ] - \frac{{σ}_{L}}{1 + {σ}_{L}} [ LE ] & ( 5 )\end{matrix}$$

where [C(i)] (g/L) is the total substrate concentration of population i including bound and free cellulose, [L] (g/L) is the concentration of total lignin, and [E] (g/L) is the concentration of total enzyme. Equations (1), (2), (3), (4) and (5) together comprise a dynamic model for enzyme adsorption.

It has been widely observed that the time required for adsorbed cellulase to reach a constant value is short relative to the time required for hydrolysis. In particular, most studies find that adsorbed enzyme reaches a constant value in less than about 90 minutes, and many studies (Boussaid et al, 1999; Chemoglazov et al, 1988; Kim et al, 1998; Lee et al, 1989; Ooshima et al, 1983; Singh et al, 1991) have found less than about 30 minutes to be sufficient. By contrast, complete hydrolysis of cellulose usually requires a day or more. Values for adsorption rate constants were determined based on the assumption that CE and LE complexes reached 95% of their equilibrium concentrations after 30 minutes. With two unknowns and two constraints ([CE]_{30 minute}=95% [CE]_{equilibrium}, [LE]_{30 minute}=95% [LE]_{equilibrium}), values of k_{ƒc} and k_{ƒl} were determined using a non-linear least square fit using Matlab. Values of adsorption parameters are presented in Table 1 along with other relevant parameters.

TABLE 1
Parameter Values

| Symbol | Value | | Source |
|---|---|---|---|
| k_{fc} | 1.8366 | L/(g h) | This patent |
| k_{fl} | 0.8359 | L/(g h) | This patent |
| k | 2.8625 | h⁻¹ | South et al, 1995 |
| e | 5.3 | | South et al, 1995 |
| c | 0.18125 | h⁻¹ | South et al, 1995 |
| K_{Cb} | 640 | h⁻¹ | Gusakov and Sinitsyn |
| K_{G} | 0.05 | g/L | Ghose and Tyagi |
| K_{C} | 1.82 | L/g | Ooshima et al, 1990 |
| K_{L} | 0.807 | L/g | Ooshima et al, 1990 |
| Kₘ | 10.56 | g/L | Phillippidis et al |
| K_{Cb/G} | 0.62 | g/L | Phillippidis et al |
| K_{C/Cb} | 5.85 | g/L | Phillippidis et al |
| K_{C/Eth} | 50.35 | g/L | Phillippidis et al |
| K_{X/Eth} | 50.0 | g/L | van Uden |
| σ_{C} | 0.0806 | | Ooshima et al, 1990 |
| σ_{L} | 0.0123 | | Ooshima et al, 1990 |
| μₘₐₓ | 0.4 | h⁻¹ | Ghose and Tyagi |
| Y_{X/G} | 0.09 | | Ghose and Tyagi |
| Y_{Eth/G} | 0.47 | | Ghose and Tyagi |

A pronounced decline in the specific reaction rate of cellulase-cellulose complexes is a well known phenomenon. As a result of this property, it is necessary to know the conversion of an individual substrate particle in order to determine its reactivity. To successfully model the case with different substrate particle ages, the model needs to account for the conversion-dependent reaction rate of each individual substrate particle population, that is, to track the extent of conversion and reactivity of individual particle populations. It is appropriate to employ particle conversion (equation 6) in the conversion-dependent reaction constant since particle conversion represents the conversion of the particle population independent of loss of particles in the reactor effluent.

$${x}_{p} ( i ) = \frac{\frac{{[ C ( i ) ]}_{0}}{{N ( i )}_{0}} - \frac{[ C ( i ) ]}{N ( i )}}{\frac{{[ C ( i ) ]}_{0}}{{N ( i )}_{0}}} = \frac{{[ C ( i ) ]}_{0} - \frac{1}{R ( i )} \times [ C ( i ) ]}{{[ C ( i ) ]}_{0}} \qquad ( 6 )$$

Where
[C]₀=g cellulose/L, fed to the reactor within one residence time
[C(i)]=g cellulose/L, population i, in the reactor @ t
[C(i)]₀=g cellulose/L, population i, fed to the reactor
N(i)₀=number of particles, population i, fed to the reactor
N(i)=number of particles, population i, in the reactor @ t

$$\begin{matrix}R ( i ) = \frac{N ( i )}{{N ( i )}_{0}} \\ = Remaining fraction of particles of \\ population i in the reactor\end{matrix}$$
n is total number of particle populations

Rate equations in the model, following the form proposed by South et al. (1995) but adapted for discrete feeding are:

$$\begin{matrix}{r}_{CE} ( i ) = ( 1 + {σ}_{C} ) \times [ {r}_{C} ( i ) + {k}_{fc} \times [ {E}_{f} ] \times ( 1 + {σ}_{C} ) \times [ {C}_{f} ( i ) ] - \frac{{k}_{fc}}{{K}_{C}} \times [ CE ( i ) ] + [ {k}_{fc} \times [ {E}_{f} ] \times ( 1 + {σ}_{C} ) \times [ {C}_{f} ( i ) ] - \frac{{k}_{fc}}{{K}_{C}} \times [ CE ( i ) ] ] & ( 7 ) \\ {r}_{C} ( i ) = - [ k \times {( 1 - {x}_{p} ( i ) )}^{e} + c ] \times \frac{[ CE ( i ) ]}{1 + {σ}_{C}} \times \frac{{K}_{C / Cb}}{[ Cb ] + {K}_{S / C}} \times \frac{{K}_{C / Eth}}{[ Eth ] + {K}_{C / Eth}} & ( 8 ) \\ {r}_{Cb} = - 1.056 \times {\sum }_{i = 1}^{n} {r}_{C} ( i ) - \frac{{K}_{Cb} \times [ Cb ] \times [ B ]}{{K}_{m} \times ( 1 + \frac{[ G ]}{{K}_{Cb / G}} ) + [ Cb ]} & ( 9 ) \\ {r}_{Xc} = \frac{[ Xc ] \times {μ}_{max} \times [ G ]}{[ G ] + {K}_{G}} \times ( 1 - \frac{[ Eth ]}{{K}_{X / Eth}} ) & ( 10 ) \\ {r}_{G} = [ - 1.056 \times {\sum }_{i = 1}^{n} {r}_{C} ( i ) - {r}_{Cb} ] \times 1.053 - \frac{{r}_{Xc}}{{Y}_{X / G}} & ( 11 ) \\ {r}_{Eth} = {r}_{Xc} \times \frac{{Y}_{Eth / G}}{{Y}_{X / G}} & ( 12 )\end{matrix}$$

Equation (7) is the rate equation for the CE complex combining the contribution from both hydrolysis and adsorption. The hydrolysis term is developed from equation (3), and the adsorption term from equation (1). Equation (8) accounts for the hydrolysis of an individual substrate particle population. Equations (9), (10), (11) and (12) represent rate equations for cellobiose, cells, glucose and ethanol, respectively.

All particle populations fed to reactor m+1 (representing either a cascade reactor or intermittent feeding to a single reactor) were considered to be one population, and an average reaction constant was calculated based on the reactivity of the entering particle populations. For a specific particle population in reactor m+1, its incremental particle conversion achieved in the reactor, x_{p,m+1}-x_{p,m}, can be normalized as

$${\tilde{x}}_{p , m + 1} = \frac{{x}_{p , m + 1} - {x}_{p , m}}{1 - {x}_{p , m}}$$

which after manipulation gives
(1−x_{p,m+1})=(1−x_{p,m})(1−\tilde{x}_{p,m+1}) (13)
For n particle populations fed at the same time into reactor m+1, the overall rate of reaction for cellulose is

$${r}_{C} = {\sum }_{i = 1}^{n} [ k \times {( 1 - {x}_{p , m + 1} ( i ) )}^{e} + c ] \times \frac{[ CE ( i ) ]}{1 + {σ}_{C}} \times S \qquad ( 14 )$$

Where

$$S = \frac{{K}_{C / Cb}}{[ Cb ] + {K}_{S / C}} \times \frac{{K}_{C / Eth}}{[ Eth ] + {K}_{C / Eth}} .$$

Substituting equation (13) into equation (14) gives

$${r}_{C} = {\sum }_{i = 1}^{n} [ k \times {( 1 - {x}_{p , m} ( i ) )}^{e} \times {( 1 - {\tilde{x}}_{p , m + 1} ( i ) )}^{e} + c ] \times \frac{[ CE ] \times w ( i )}{1 + {σ}_{C}} \times S \qquad ( 15 )$$

Where w(i) is the fraction of concentration of cellulose enzyme complex of population i, and

$${\sum }_{i = 1}^{n} w ( i ) = 1.$$

For the particles fed into reactor m+1 at the same time, we assume they have the same normalized incremental particle conversion since they have the same age in the reactor
\tilde{x}_{p,m+1}(1)=\tilde{x}_{p,m+1}(2)=. . . =\tilde{x}_{p,m+1}(n)=\tilde{x}_{p,m+1} (16)
Substituting equation (16) into equation (15) gives

$$\begin{matrix}{r}_{C} = \{ [ {\sum }_{i = 1}^{n} k \times {( 1 - {x}_{p , m} ( i ) )}^{e} \times w ( i ) ] \times {( 1 - {\tilde{x}}_{p , m + 1} )}^{e} + c \} \times \\ \frac{[ CE ]}{1 + {σ}_{C}} \times S \\ = \{ {k}^{′} \times {( 1 - {\tilde{x}}_{p , m + 1} )}^{e} + c \} \times \frac{[ CE ]}{1 + {σ}_{C}} \times S\end{matrix} \qquad ( 17 )$$

Where

$${k}^{′} = [ {\sum }_{i = 1}^{n} k \times {( 1 - {x}_{p , m + 1} ( i ) )}^{e} \times w ( i ) ] ,$$

is the average remaining hydrolysis rate constant for the older particle populations at the time of a feeding. Using this model, it was possible to predict the experimental results shown in FIG. 3 and to explain the observed behavior.

As feeding frequency increases, the system more asymptotically approaches the fully continuous state. Particles begin to leave the reactor immediately after they are fed in a continuously stirred tank reactor (CSTR), whereas all particles react for a minimum time in the case of intermittently-fed reactors. Therefore, the increased efficiency of semi-continuously operated reactors can be attributed to increased substrate reaction time for newly introduced, unreacted substrate particles. The specific activity of cellulase-cellulose complexes decreases with time, so unreacted substrate has the highest potential for conversion and loss of unreacted substrate (which occurs in a CSTR) results in a lower mean conversion achieved at a given enzyme loading.

It is to be understood that various modifications can be made in the present systems and methods. For example, the fermentation reaction may be run using types of organisms which are not specifically disclosed herein. In addition, while the general design of a suitable bioreactor is provided herein, various modifications and refinements of the bioreactor can be made.

All references and publications cited herein are expressly incorporated by reference herein.

We claim:

1. A method for producing ethanol from a cellulosic substrate, comprising the steps of:
(a) roviding within a reaction vessel, a reaction mixture in the form of a slurry comprising cellulosic substrate, an enzyme and a fermentation agent, wherein the reaction mixture is reacted under conditions sufficient to initiate and maintain a fermentation reaction;
(b) etermining, by experiment or theoretical calculations, an optimum feeding frequency; and
(c) roviding additional quantities of the cellulosic substrate and the enzyme into the reaction vessel at an interval(s) according to the optimized feeding frequency.

2. The method of claim 1, wherein the optimized feeding frequency is in a range of about 1 to 8.

3. The method of claim 1 further comprising the step of removing from the reaction vessel an ethanol-containing effluent.

4. The method of claim 1 wherein the cellulosic substrate comprises woody biomass, forage grasses, herbaceous energy crops, agricultural residue, waste paper sludge and municipal solid waste.

5. The method of claim 1 wherein the ethanol-containing effluent is removed from the top portion of the reaction vessel.

6. The method of claim 1 wherein the slurry is agitated by mechanical mixing.

7. The method of claim 1 wherein the fermentation agent is selected from fungi and bacteria.

8. The method of claim 1 wherein the enzyme is cellulase.

9. The method of claim 1 being carried out using a semi-continuously solids-fed bioreactor.

10. A method of operating a reactor system to provide semi-continuous solids-fed fermentation of paper sludge, comprising:
advancing a paper sludge under a semi-continuous controlled flow rate;
chopping the paper sludge as it advances to subdivide the paper sludge into comparatively smaller particles;
digesting the comparatively smaller particles in an agitator by the action of a hydrolyzing enzyme; and
controlling the flow rate and amount of hydrolyzing enzyme added to the agitator for use in the step of digesting to establish a semi-continuous state of flow through the reactor system over an extended period of time while also maintaining a predetermined conversion efficiency of the paper sludge by action of the hydrolyzing enzyme at an optimized loading concentration.

11. The method of claim 10, wherein the optimized loading concentration is in a range of about 5 to 15 fpu/g.

12. The method of claim 10 wherein the hydrolyzing enzyme is cellulase.

13. The method of claim 10 further comprising the step of adding a fermentation agent selected from fungi and bacteria.

14. The method of claim 13 further comprising the step of removing from the agitator an ethanol-containing effluent.

15. The method of claim 14 wherein the ethanol-containing effluent is removed from the top portion of the agitator.

16. A semi-continuous solids-fed bioreactor comprising:
a feed tube containing a plug of solid waste;
a screw driven by a timer-controlled motor, the screw acting to advance the plug of solid waste into a chopper; and
an agitator that receives solid waste particles from the chopper and enzymes, fermentation agents and liquid reagents from an inlet valve, the solid waste particles, enzymes, fermenation agents and liquid reagents forming a slurry in the agitator, wherein the agitator includes a mixer driven by a second motor and at least one conduit from which products may be removed.

17. The bioreactor of claim 16, further comprising an isolation valve connecting the feed tube and the chopper.

18. The bioreactor of claim 17, wherein the isolation valve comprises a ball valve.
